(19) United States
(12) Patent Application Publication
(10) Pub. No.: US 2026/0355163 A1
(43) Pub. Date: Nov. 12, 2026

(54) TREATMENT OF FASCIOLOSIS

(71) Applicant: INTERVET INC., Rahway, NJ (US)

(72) Inventors: Michael BERGER, Wiesbaden (DE); Ulrich SONDERN, Dortmund (DE)

(73) Assignee: INTERVET INC., Rahway, NJ (US)

(21) Appl. No.: 19/107,201

(22) PCT Filed: Sep. 1, 2023
(86) PCT No.: PCT/EP2023/074068

§ 371 Date: Feb. 27, 2025

(30) Foreign Application Priority Data
Sep. 2, 2022 (EP) 22193586.9

(51) Int. Cl. A61K 31/422; A61K 31/4155; A61K 45/06; A61P 33/10

(52) U.S. Cl. CPC A61K 31/422; A61K 31/4155; A61K 45/06; A61P 33/10

(57) ABSTRACT

The present invention relates to compounds and compositions of Formula (I) for treating a fasciolosis in a mammal.

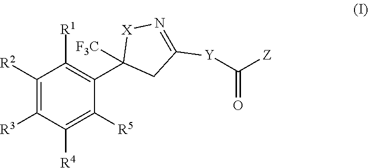

### FIELD OF THE INVENTION

The present invention relates to compounds and compositions for treating fasciolosis in a mammal.

### BACKGROUND OF THE INVENTION

Fasciolosis is an important zoonotic disease that is responsible for a significant loss in food resource and animal productivity. Fasciolosis is a disease of both veterinary and public health importance, infecting over 600 million animals. The infection causes massive loss through liver condemnation, reduced production of milk, meat, and wool, veterinary care, metabolic disease as well as mortality

Fasciolosis is caused by the trematode parasite Fasciola spp. Fasciola hepatica and Fasciola gigantica are the two main species responsible for most fasciolosis cases in mammals. Fasciola hepatica is cosmopolitan in nature because of its ability to infect a variety of species, while Fasciola gigantica is more common in tropical countries, though both species are found in Africa and Asia.

Fasciolosis can result from the migration of large numbers of immature fluke larvae through the liver, from the presence of adult flukes in the bile ducts, or both. Fasciolosis occurs in three main clinical forms—acute, subacute and chronic fasciolosis. Which form occurs depends on the numbers of infective metacercariae ingested and the period over which they are ingested.

The primary impact of fasciolosis is on farmed ruminants, however, they are not selective regarding the final host and successfully infect a wide range of mammals, including humans. Among the farmed ruminants, fasciolosis may cause annual economic losses of several billions of dollars world-wide.

Mature F. hepatica are large, leaf-shaped trematodes; about 3 to 5 cm in length and 1 cm in width. Both the immature and the mature stages of the parasite feed by secreting enzymes, most notably cysteine proteases, which break down blood and the liver tissue. The parasites irritate the walls of the bile ducts causing hyperplasia of the bile duct epithelium.

There is a range of products that can be used to treat mammals against fasciolosis, especially ruminants such as sheep and cattle. When recommending treatment plans it is important to use drugs that target the stage of Fasciola spp. most likely to be present within the diseased animal at the time, to help reduce selection pressure.

From the available flukicidal compounds only one compound, triclabendazole (TCBZ), is effective against mature stages of the common liver fluke (Fasciola hepatica) as well as all immature stages, i.e. early immature larvae (1 to 6 weeks old) and immature larvae (6 to 9 weeks old). In fact, it is the only flukicide effective against all early immature liver fluke larvae.

However, resistance to flukicides can occur with repeated and frequent use, alternatives should be used wherever possible, particularly in late winter and spring, to reduce potential for the development of TCBZ-resistance.

Other drugs for treating Fasciola-infected livestock are albendazole, closantel, nitroxynil and oxyclozanide. However, all of them are only effective against the mature flukes and not against the immature stages, such as early immature larvae (1 to 6 weeks old) and immature larvae (6 to 9 weeks old). Closantel is partially effective against 3-8 weeks old flukes and fully effective against mature flukes; nitroxynil is effective against fluke from 8 weeks post infection and clorsulon, oxyclozanide and albendazole are effective only against mature flukes.

Consequently, current treatment options suffer from the ineffectiveness of most compounds against immature flukes during the migratory phase, the phase in which the most severe damage occurs to liver tissue when migrating towards the bile ducts.

The upcoming resistance to triclabendazole and other flukicidal compounds and lack of effective substitutes creates a pressing need in the field for alternative treatment options.

It is an object of this invention to overcome at least one of the above-referenced problems.

Isoxazoline compounds are known in the art and these compounds are described, for example, in US patent application US 2007/0066617, and international patent applications WO 2005/085216, WO 2007/079162, WO 2009/002809, WO 2009/024541, WO 2009/003075, WO 2009/080250, WO 2010/070068 and WO 2010/079077.

This class of compounds is known to possess excellent activity against ectoparasites i.e. parasites that have their permanent or temporary habitat on the external surface of animals, such as parasitic insects and acarids such as ticks and fleas and mites.

However, efficacy of isoxazoline compounds to treat fasciolosis has not been reported in prior art.

### SUMMARY OF THE INVENTION

The Applicant has addressed the problems of the prior art by identifying compounds that are effective at treating fasciolosis.

The current invention provides a compound of Formula (I)

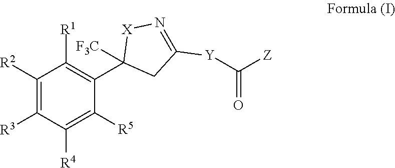

R¹-R⁵ is H, F, Cl or CF₃
X is O or S, preferably O
Y is selected from

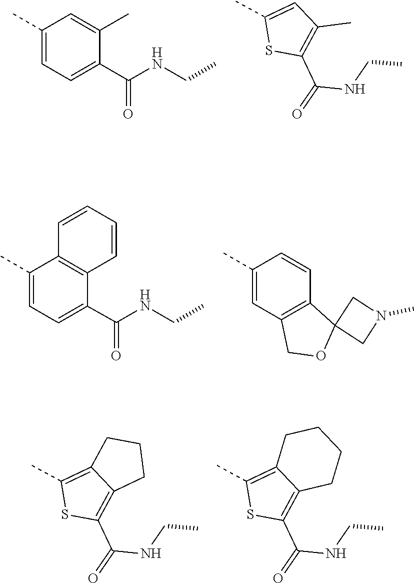

wherein
[symbol] denotes the bond to the isoxazoline or isothiazoline
[symbol] denotes the bond to the carbonyl group
Z is selected from

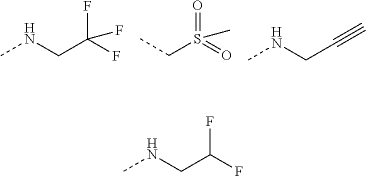

wherein
[symbol] denotes the bond to the carbonyl group
for use in the treatment of fasciolosis of mammals.

Another aspect of the current invention is a composition comprising such compound and a pharmaceutically acceptable carrier for use in the treatment of fasciolosis of mammals.

In an alternative embodiment the compound for use according to the current invention is tigolaner.

### DETAILED DESCRIPTION OF THE INVENTION

The current inventors found that compounds of Formula (I) are addressing the problem by providing a new fasciolosis treatment option.

It has been found that these compounds are particularly effective against immature liver flukes, that is fluke less than 9 weeks old, from mammals, whereas most commercially available fasciolicides are more effective against mature liver fluke, that is, fluke more than 9 weeks old, and indeed many such available compounds are barely effective against immature fluke.

Accordingly, use of the compounds of Formula (I) provides a valuable complementary treatment of fasciolosis when used alone or when used in conjunction with existing remedies.

### DEFINITIONS AND GENERAL PREFERENCES

When used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to any or more of that entity. As such, the terms “a” (or “an”), “any or more,” and “at least one” are used interchangeably herein.

The term “fasciolosis” refers to a clinical or subclinical disease of mammals in connection with an infection of a mammal with Fasciola hepatica or Fasciola gigantica.

In one embodiment the fasciolosis is due to liver fluke infestation by immature or early immature stage and symptoms caused by such infestation.

Fascioliosis ranges in severity from a devastating disease in sheep, and pseudo-ruminants such as alpacas, and llamas to a subclinical infection that is more common in cattle.

Acute disease occurs 2-6 weeks after the ingestion of large numbers of metacercaria over a short period. In sheep, acute fascioliosis occurs seasonally and is manifest by a distended, painful abdomen; anemia; and sudden death. The acute syndrome can be complicated by concurrent infections with Clostridium novyi, resulting in black disease (clostridial necrotic hepatitis).

In subacute disease, large numbers of metacercaria are ingested over longer periods of time; survival is longer (7-10 weeks), even in cases with extensive hepatic damage, but deaths occur because of hemorrhage and anemia. Subacute fasciolosis is characterized by jaundice, some ill thrift and anemia. The burrowing fluke stages causes extensive tissue damage, leading to hemorrhaging and liver damage. The outcome is severe anemia, liver failure and death.

Chronic fascioliosis can occur in any season, but it manifests primarily in late fall and winter. It occurs as a result of ingesting moderate numbers of metacercariae over much longer periods of time.

Chronic fasciolosis is the most common form of liver fluke infection in sheep, goats and cattle—and particularly in more resistant hosts, such as horses and pigs. It occurs when the parasites reach the bile ducts in the liver. The fluke ingests blood, which produces severe anemia and chronic inflammation and enlargement of the bile ducts. The clinical signs develop slowly. The animals become increasingly anemic, appetite is lowered, the mucous membranes of the mouth and eyes become pale, and some animals develop oedema under the jaw (‘bottle jaw’). Affected animals are reluctant to travel.

Significant production losses caused by (especially subclinical) fasciolosis in livestock, include reduced production and quality of wool, reduced lambing percentages, poor growth rate of lambs and/or increased costs for replacement stock in sheep; reduced calving percentages, poor growth rate of calves and/or increased costs for replacement stock in cattle; reduced production and quality of milk in dairy cattle and lower growth rates and lower feed conversion rates in fattening cattle.

The term “liver fluke infection” refers to infestation of a mammal with Fasciola hepatica or Fasciola gigantica parasites. Unless otherwise indicated the term includes infestation or infection by flukes at any stage of maturity and mixtures of stages of maturity.

The liver fluke has a complex life cycle. In its final host, Fasciola spp. can be categorized into three stages, early immature, immature, and mature (commonly termed the adult stage).

In one embodiment the fasciolosis is due to liver fluke infection by early immature stage liver fluke i.e., by early immature larvae (1 to 6 weeks old).

In another embodiment the fasciolosis is due to liver fluke infection by immature stage liver fluke i.e., by immature larvae (6 to 9 weeks old).

In another embodiment the fasciolosis is due to liver fluke infection by mature stage liver fluke i.e., by mature flukes (>9 weeks old).

In a preferred embodiment the compound for use according to the invention is effective against all immature liver fluke larvae.

In a more preferred embodiment, the compound for use according to the invention is effective against all stages of liver flukes.

The term “mammal” refers to warm-blooded vertebrate animals having hair or fur and secrete milk by the females for the nourishment of the young and includes humans. Particular mammals are ruminants, for example cattle, sheep, goats, bison, African buffalo, water buffalo, antelopes, deer, moose, elks, and giraffes.

Also, included in the term mammal are the so-called pseudo-ruminants, for example, llamas and alpacas.

Also, included in the term mammal are other susceptible animals such as horses, pigs and donkeys.

More particularly, mammals are understood to be sheep, goats, bison, African buffalo, water buffalo, and cattle. Even more particularly, mammals are sheep and cattle.

In one embodiment, the mammal is a ruminant. In a preferred embodiment, the mammal is selected from the group consisting of sheep and cattle. In one embodiment, the ruminant is sheep. In another embodiment, the ruminant is cattle.

According to the present invention the term “veterinary” has the same definition as “pharmaceutical” but adapted to mammal animals (meaning non-human beings).

More precisely, a “veterinary drug” (or medicine or composition) means a compound or mixture of compounds (conventionally together with inert formulation ingredients), which is used, or is manufactured, sold or represented as suitable for use, in the diagnosis, treatment, control, eradication, mitigation or prevention of disease or abnormal physical or mental state or the symptoms thereof in an animal; or restoring, correcting, controlling, or modifying any physical, mental or organic function in an animal.

The ‘treatment’, and similar terms such as ‘treating’ or ‘treat’ as used herein, refer to the administration of an effective amount of the compound as described for use in the invention to a mammal which has been diagnosed with and/or shows symptoms of fasciolosis of more or less severity.

The skilled clinician can readily determine a mammal in need of the present treatment. The typical symptoms of acute, subacute and chronic fasciolosis have been described earlier. The fasciolosis as cause of the symptoms can be detected by antibody testing, specifically ELISA testing, in particular of the blood and milk.

The fasciolosis as cause of the symptoms by detection of the Fasciola spp. infection can also be diagnosed by examining fecal specimens, including by ELISA testing. Moreover, modeling based on season, rainfall, temperature, and other local conditions can be used to predict times when fasciolosis is more likely.

Preferably the fasciolosis is treated during autumn, winter, or early spring season.

Autumn is the time of year when grazing mammals are most likely to ingest large numbers of liver fluke metacercariae on the pasture. In Australia this is during the months April/May. Once ingested liver fluke larvae migrate through the liver causing damage to the liver tissue and blood loss.

This high-risk situation extends (e.g., depending on the weather conditions) in many situations to the winter and early spring season. Therefore another treatment can be made during late winter/early spring, this is in Australia during the months August/September.

Therefore, it is important that a compound allows effective treatment of the liver fluke stages present at this high-risk period. These are all stages of flukes, but especially important is the activity against immature larvae stages, especially early immature larvae that are present during this time.

In one embodiment the compounds for use according to the invention are especially useful—in a situation wherein fasciolosis of the mammal—especially cattle or sheep—is due to an immature stage liver fluke infection and such infection is treated during autumn, winter, or early spring season. This provides a very useful treatment option to treat fasciolosis and the symptoms of such disease during the season with especially high risk of damage to the infected animal. During such season the immature stages of liver fluke are predominantly.

In another embodiment the compound for use according to the current invention is used for treatment during summer, sometimes called mid-season treatment in which predominantly adult stages of liver flukes and symptoms caused by this infection are present in the treated mammal. In Australia this is done during the months January/February.

What constitutes an “effective amount” for use in the invention is the amount, therapeutic dose, or quantity of a compound as described herein, that is required for a significant reduction of the parasites infesting such animal and when administered to a mammal for treating a disease, is sufficient to ameliorate or prevent the symptoms of acute, subacute or chronical fasciolosis to effect such treatment for the disease.

The “therapeutically effective amount” for the treatment of the different forms of fasciolosis will vary depending on the specific compound, the veterinary drug form, the age, weight, etc., of the mammal to be treated.

Compounds of Formula (I) for use in the current invention are:

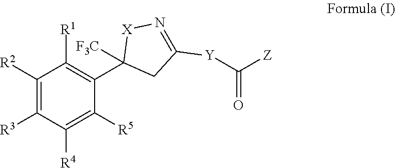

R¹-R⁵ is H, F, Cl or CF₃
X is O or S, preferably O
Y is selected from

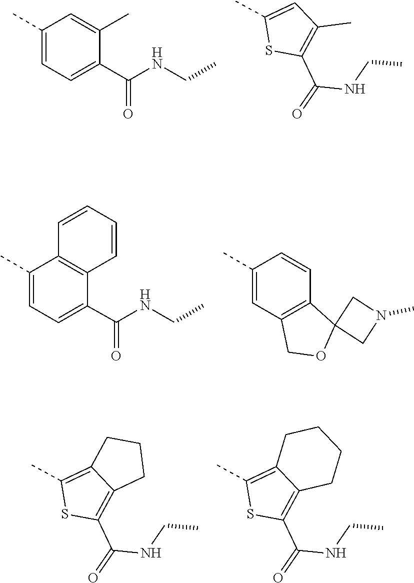

wherein
[symbol] denotes the bond to the isoxazoline or isothiazoline
[symbol] denotes the bond to the carbonyl group
Z is selected from

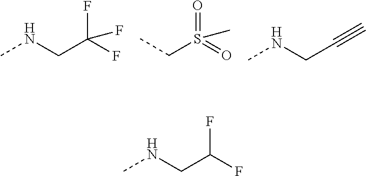

wherein
[symbol] denotes the bond to the carbonyl group.

In one preferred embodiment of the invention and/or embodiments thereof the compound is 4-[5-(3,5-dichlorophenyl)-5-trifluoromethyl-4,5-dihydroisoxazol-3-yl]-2-methyl-N-[(2,2,2-trifluoro-ethylcarbamoyl)-methyl]-benzamide (CAS RN 864731-61-3). This compound is also known as fluralaner.

In one preferred embodiment of the invention and/or embodiments thereof the compound is 4-[5-[3-chloro-5-(trifluoromethyl)phenyl]-4,5-dihydro-5-(trifluoromethyl)-3-isoxazolyl]-N-[2-oxo-2-[(2,2,2-trifluoroethyl)amino]ethyl]-1-naphthalene-carboxamide (CAS RN 1093861-60-9). This compound is also known as a 4-[5-(5-chloro-α,α,α-trifluoro-m-tolyl)-4,5-dihydro-5-(trifluoromethyl)-1,2-oxazol-3yl]-N-[2-oxo-2-[(2,2,2-trifluoroethylamino]ethyl]naphthalene-1-or afoxolaner. Afoxolaner is for example disclosed in WO 2007/079162. In one embodiment the isoxazoline compound is Esafoxolaner.

In one especially preferred embodiment of the invention and/or embodiments thereof the compound is 1-(5′-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethan-1-one, preferably 1-(5′-((5S)-(5-(3,5-dichloro-4-fluorophenyl)-5-(trifluoromethyl)-4,5-dihydroisoxazol-3-yl)-3′H-spiro[azetidine-3,1′-isobenzofuran]-1-yl)-2-(methylsulfonyl)ethan-1-one (CAS RN: 1398609-39-6). This compound is known as sarolaner.

In one preferred embodiment of the invention and/or embodiments thereof the compound is 3-methyl-N-(2-oxo-2-((2,2,2-trifluoroethyl)amino)ethyl)-5-[5-(3,4,5-trichlorophenyl)-5-(trifluoromethyl)-4,5-dihydroisoxazol-3-yl]thiophene-2-carboxamide, preferably methyl-N-(2-oxo-2-((2,2,2-trifluoroethyl)amino)ethyl)-5-[(5S)-5(3,4,5-trichlorophenyl)-5-(trifluoromethyl)-4,5-dihydroisoxazol-3-yl]thiophene-2-carboxamide (CAS RN: 1369852-71-0). This compound is known as lotilaner.

In one preferred embodiment of the invention and/or embodiments thereof a compound used as alternative is 2-chloro-N-(1-cyanocyclopropyl)-5-[1-[2-methyl-5-(1,1,2,2,2-pentafluoroethyl)-4-(trifluoromethyl)pyrazol-3-yl]pyrazol-4-yl]benzamide (CAS RN 1621436) This compound is known as tigolaner.

In one preferred embodiment of the invention and/or embodiments thereof the compound is 4H-Cyclopenta[c]thiophene-1-carboxamide, 3-[(5S)-5-(3,5-dichloro-4-fluorophenyl)-4,5-dihydro-5-(trifluoromethyl)-3-isoxazolyl]-N-[2-[(2,2-difluoroethyl)amino]-2-oxoethyl]-5,6-dihydro- (CAS 1414642-93-5). This compound is known as mivorilaner.

In one preferred embodiment of the invention and/or embodiments thereof the compound of Formula (I) is (Z)-4-[5-(3,5-dichlorophenyl)-5-trifluoromethyl-4,5-dihydroisoxazol-3-yl]-N-[(methoxy-imino)methyl]-2-methylbenzamide (CAS RN 928789-76-8).

In one preferred embodiment of the invention and/or embodiments thereof the compound is 4-[5-(3,5-dichlorophenyl)-5-(trifluoromethyl)-4H-isoxazol-3-yl]-2-methyl-N-(thietan-3-yl) benzamide (CAS RN 1164267-94-0) that was disclosed in WO 2009/0080250.

In one preferred embodiment of the invention and/or embodiments thereof the compound is 5-[5-(3,5-dDichlorophenyl)-4,5-dihydro-5-(trifluoromethyl)-3-isoxazolyl]-3-methyl-N-[2-oxo-2-[(2,2,2-trifluoroethyl) amino]ethyl]—2-thiophenecarboxamide (CAS RN 1231754-09-8) that was disclosed in WO 2010/070068.

In one preferred embodiment of the invention and/or embodiments thereof the compound of Formula (I) is selected from fluralaner (including (S) fluralaner), afoxolaner (including esafoxolaner), sarolaner, or lotilaner.

Especially preferred is fluralaner (corresponding to 4-[5-(3,5-dichlorophenyl)-5-trifluoromethyl-4,5-dihydroisoxazol-3-yl]-2-methyl-N-[(2,2,2-trifluoro-ethylcarbamoyl)-methyl]-benzamide) as systemic insecticide and/or acaricide (a) which is represented by Formula (II)

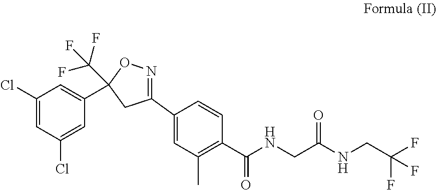 [SMILES: Cc1cc(C2=NOC(c3cc(Cl)cc(Cl)c3)(C(F)(F)F)C2)ccc1C(=O)NCC(=O)NCC(F)(F)F]

The compounds for use according to the current invention may exist in various isomeric forms. A reference to a compound always includes all possible isomeric forms of such a compound. Unless otherwise stated, a compound structure that does not indicate a particular conformation is intended to encompass compositions of all the possible conformational isomers of the compound, as well as compositions comprising fewer than all the possible conformational isomers.

Compounds of Formula (I) can be prepared according to one or other of the processes described e.g., in patent applications US 2007/0066617, WO 2007/079162, WO 2009/002809, WO 2009/080250, WO 2010/070068, WO 2010/079077, WO 2011/075591 and WO 2011/124998 or any other process coming within the competence of a person skilled in the art who is an expert in chemical synthesis.

For the chemical preparation, a person skilled in the art is regarded as having at her/his disposal, inter alia, the entire contents of “Chemical Abstracts” and of the documents cited therein.

This invention is also directed to composition for use according to the invention comprising more than one pharmaceutically active ingredient, i.e. wherein the composition for oral administration comprises another active pharmaceutical ingredient.

As used herein, the term “comprise,” or variations thereof such as “comprises” or “comprising,” are to be read to indicate the inclusion of any recited integer (e.g., a feature, element, characteristic, property, method/process step or limitation) or group of integers (e.g., features, element, characteristics, properties, method/process steps or limitations) but not the exclusion of any other integer or group of integers.

Thus, as used herein the term “comprising” is inclusive or open ended and does not exclude additional, unrecited components or method/process steps.

The term “in combination with” as well as “combination” and “synergistic combination” as used herein are taken to mean that a compound different from a compound of Formula (I) or another compound of Formula (I) is administered prior to, during, or after the administration of an compound of Formula (I). That is, in these are administered either sequentially or simultaneously to the mammal. In a preferred embodiment the combination is with a compound different from a compound of Formula (I).

Typically, sequentially administration means administration of within three days of administration of the other.

More typically, the compounds for use of the current invention are administered together with the combination compound within a day of each other and even more typically on the same day.

Sequential administration also means administration of such compounds within an hour, or even minutes, of administration of the other compound.

Simultaneous administration includes preferably the presence of such compounds in the same veterinary product.

Those of ordinary skill in the veterinary pharmaceutical arts will be entirely familiar with the identity of such active ingredients which may include, without limitation antiparasitics such as endoparasiticides (including anthelmintics) and ecto-parasiticides, hormones and/or derivatives thereof, anti-inflammatory compounds and minerals and vitamins.

Especially preferred is a combination with selenium, especially as sodium selenate.

The active ingredients are preferably antiparasitics, more preferably endoparasiticides, preferably anthelmintics and are preferably selected from the group consisting of avermectins (e.g., ivermectin, doramectin, abamectin, eprinomectin); milbemycins (moxidectin and milbemycin oxime); pro-benzimidazoles (e.g., febantel, netobimin, and thiophanate); benzimidazole derivatives, such as a thiazole benzimidazole derivatives (e.g., thiabendazole and cambendazole), carbamate benzimidazole derivatives (e.g., fenbendazole, albendazole (oxide), mebendazole, oxfendazole, parbendazole, oxibendazole, flubendazole, and triclabendazole); imidazothiazoles (e.g., levamisole and tetramisole); tetrahydropyrimidine (morantel and pyrantel), salicylanilides (e.g., closantel, oxyclozanide, rafoxanide, and niclosamide); nitrophenolic compounds (e.g., nitroxynil and nitroscanate); benzenedisulfonamides (e.g., clorsulon); pyrazinoisoquinolines (e.g., praziquantel and epsiprantel); heterocyclic compounds (e.g., piperazine, diethylcarbamazine, and phenothiazine); dichlorophen, arsenicals (e.g., thiacetarsamide, melorsamine, and arsenamide); cyclooctadepsipeptides (e.g., emodepside); paraherquamides (e.g. derquantel); and amino-acetonitrile compounds (e.g. monepantel); amidine compounds (e.g., amidantel and tribendimidin), including all pharmaceutically acceptable forms, such as salts, solvates or N-oxides.

Especially preferred are combinations with ivermectin, eprinomectin, moxidectin, levamisole, fenbendazole, abamectin, monepantel, derquantel, oxfendazole, albendazole, closantel and combinations thereof.

An effective amount of the compound for use according to the current invention is administered to the mammal orally or parenterally.

In one embodiment, an effective amount is administered orally to the mammal.

In one embodiment, an effective amount is administered parenterally to the mammal.

Generally, the compounds are administered to an animal in need thereof in the form of a pharmaceutical or veterinary composition.

Such compositions for oral administration include components conventionally used in the art of formulation and may therefore be processed in a known manner to give, for example, solutions, emulsions, soluble powders, powder mixtures, granules or microencapsulation in polymeric substances.

Such formulations, preparations or compositions optionally include a solid or liquid adjuvant, and are produced in a manner well-known in the art, for example by intimately mixing and/or grinding the active ingredients with the adjuvants, for example with solvents, solid carriers, etc.

Oral administration can also be accomplished by drench, gavage, tablet, capsule, or in feed.

A number of veterinary compositions are known to be suitable for oral administration to animals, but they vary for the different animal species. Oral administration can be accomplished by conventional dosage forms, such as e.g. drench, gavage, tablet, capsule, or in feed.

For sheep conventionally pharmaceutically active ingredients are administered orally as solids (e.g., tablets or boluses) or liquids, and via their feed or drinking water. In large sheep flocks the use of oral drenches is the most common oral dosage form.

Drenching means that a liquid, potentially slightly viscous pharmaceutical composition comprising the active pharmaceutical ingredient and excipients is applied via the mouth with a specific drenching gun that dispenses the composition into the sheep's throat.

Oral drench compositions are in general solutions or suspensions and generally not more than 20 ml, preferably not more than 15 ml of such drench are applied per sheep.

In one embodiment a concentrated solution is used to administer the compound for use according to the invention via drench application to sheep.

In another embodiment a suspension formulation is used for the drench application.

Excipients may comprise conventional non active pharmaceutically and veterinarily acceptable components or excipients e.g. fillers, binders, flavoring agents, solvents, colorants, glidants, preservatives, viscosity enhancer, surfactants, stabilizers, such as antioxidants.

The pharmaceutical excipients are excipients with which the person skilled in the art is familiar, such as those which are described in the European Pharmacopoeia.

Parenteral administration includes injectable administration, especially subcutaneous administration. This is a preferred administration route for larger ruminant animals such as cattle.

A number of veterinary compositions are known to be suitable for subcutaneous injection administration to animals, generally liquid compositions such as solutions or suspensions of the compounds of Formula (I) using pharmaceutically acceptable solvents and/or suspending agents.

The composition for subcutaneous administration can be either a liquid solution or suspension or a semi-solid or solid composition e.g., an implant.

A solution is a mixture of two or more components that form a single phase that is homogeneous down to the molecular level. A suspension consists of insoluble solid particles dispersed in a liquid medium, with the solid particles accounting for about 0.5% to about 30% of the suspension. The liquid may be aqueous, oily, or both.

An example for a suitable injectable solution of compounds, especially fluralaner, is a pharmaceutical composition that comprises an isoxazoline compound, and a pharmaceutically acceptable carrier comprising 2 pyrrolidone, PEG and propylene glycol. An alternative is an aqueous suspension.

The amounts of each of the components in the final product may be varied considerably, depending upon the nature of the pharmaceutically active ingredients, the weight and condition of the subject treated, and the unit dosage desired.

In general the effective dose of the compound of formula (I) is between 0.5 mg/kg bodyweight and 50 mg/kg bodyweight. Those of ordinary skill in the art will be able to adjust dosage amounts for particular compounds in the composition in light of the teachings of this disclosure and the available information about such compounds.

The product according to the current invention conventionally further comprises physiologically acceptable formulation excipients known in the art e.g., as described in “Gennaro, Remington: The Science and Practice of Pharmacy” (20th Edition, 2000) incorporated by reference herein.

All such ingredients, carriers and excipients must be substantially pharmaceutically or veterinary pure and non-toxic in the amounts employed and must be compatible with the pharmaceutically active ingredients.

The veterinary composition for use according to the invention can be in one embodiment mixed with an existing commercial product before administration to an animal.

In one embodiment the composition includes a combination of an effective amount of a compound for use according to the current invention and another compound (or a commercial product with such compound) that controls a different parasite infestation, e.g., against parasitic helminths or alternatively ectoparasites, especially fly strike. In a specific embodiment two compounds of Formula (I) as described above are included in a composition that control different parasites.

### EXAMPLES

The invention will now be described with reference to specific Examples. These are merely exemplary and for illustrative purposes only: they are not intended to be limiting in any way to the scope of the monopoly claimed or to the invention described.

### Example

### Material and Methods

The effect of test compounds in a whole organism screen of Fasciola hepatica Newly Excysted Juveniles (NEJs) was investigated along with DMSO (− control), triclabendazole (10 μM, + control) and media) and their anti-flukicidal activity was assessed at both 24 and 72 hours.

Compounds were tested at 10 μM and/or in declining concentrations of the test compound of 25 μM, 12.5 μM, 6.25 μM, 3.125 μM, and 1.562 μM.

TABLE 1
Test compounds

| Compound | | |
|---|---|---|
| Number | INN | Structure |
| 1 | fluralaner | 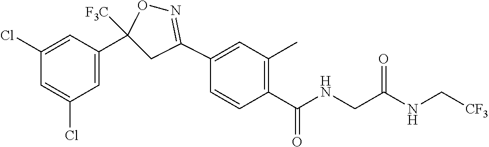 [SMILES: Cc1cc(C2=NOC(c3cc(Cl)cc(Cl)c3)(C(F)(F)F)C2)ccc1C(=O)NCC(=O)NCC(F)(F)F] |
| 2 | (S)-fluralaner | 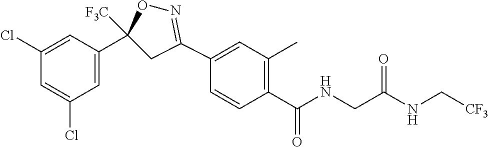 [SMILES: Cc1cc(C2=NO[C@@](c3cc(Cl)cc(Cl)c3)(C(F)(F)F)C2)ccc1C(=O)NCC(=O)NCC(F)(F)F] |
| 3 | afoxolaner | 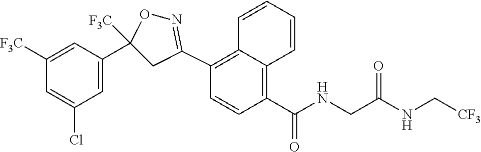 [SMILES: O=C(CNC(=O)c1ccc(C2=NOC(c3cc(Cl)cc(C(F)(F)F)c3)(C(F)(F)F)C2)c2ccccc12)NCC(F)(F)F] |
| 4 | lotilaner | 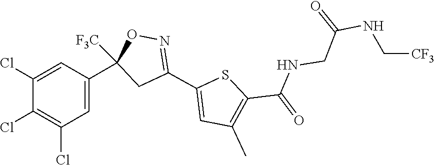 [SMILES: Cc1cc(C2=NO[C@@](c3cc(Cl)c(Cl)c(Cl)c3)(C(F)(F)F)C2)sc1C(=O)NCC(=O)NCC(F)(F)F] |
| 5 | sarolaner | 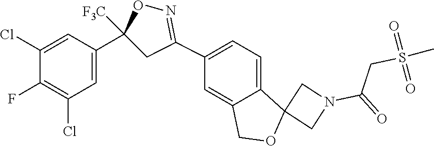 [SMILES: CS(=O)(=O)CC(=O)N1CC2(C1)OCc1cc(C3=NO[C@@](c4cc(Cl)c(F)c(Cl)c4)(C(F)(F)F)C3)ccc12] |
| 6 | | 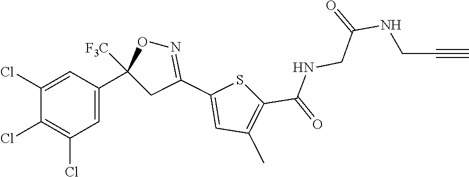 [SMILES: C#CCNC(=O)CNC(=O)c1sc(C2=NO[C@@](c3cc(Cl)c(Cl)c(Cl)c3)(C(F)(F)F)C2)cc1C] |
| 7 | | 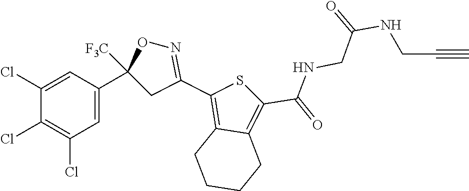 [SMILES: C#CCNC(=O)CNC(=O)c1sc(C2=NO[C@@](c3cc(Cl)c(Cl)c(Cl)c3)(C(F)(F)F)C2)c2c1CCCC2] |
| 8 | | 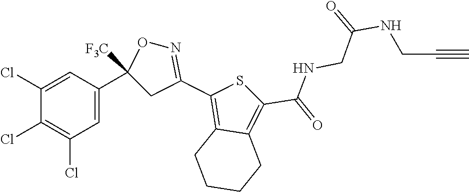 [SMILES: C#CCNC(=O)CNC(=O)c1sc(C2=NO[C@](c3cc(Cl)c(Cl)c(Cl)c3)(C(F)(F)F)C2)c2c1CCCC2] |
| 9 | | 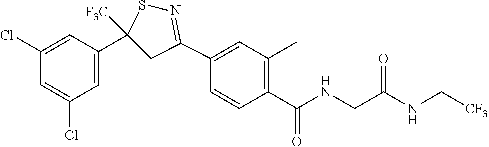 [SMILES: Cc1cc(C2=NSC(c3cc(Cl)cc(Cl)c3)(C(F)(F)F)C2)ccc1C(=O)NCC(=O)NCC(F)(F)F] |
| 10 | tigolaner | 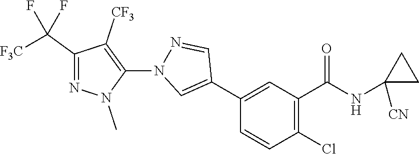 [SMILES: Cn1nc(C(F)(F)C(F)(F)F)c(C(F)(F)F)c1-n1cc(-c2ccc(Cl)c(C(=O)NC3(C#N)CC3)c2)cn1] |

The viability detection of Fasciola hepatica NEJs is based on qualitative microscopic evaluation of motility and phenotype and additionally measuring propidium iodide to make use of the differential uptake of fluorophores (such as propidium iodide) by living organisms.

NEJs were prepared according to Edwards et al. [1]. Approximately 12 NEJs/compound were cultivated. NEJ/compound co-cultures were placed at 37 °C in a humidified atmosphere containing 5% CO₂. NEJ/compound co-cultures were incubated for a total of 72 hrs. At 24h, an assessment of the gross phenotype was made for all NEJ/compound co-cultures and bright field images were captured. At 72 h both phenotype and motility were quantified in comparison to live DMSO controls based on a microscopic scoring method detailed in [1] as well as summarised in Table 1 below.

Propidium Iodide (PI) was then added to the NEJ/compound co-cultures (to a final concentration of 2 ag/mL) and these co-cultures were imaged under both Bright field and Texas Red settings.

### Results:

The following compounds were found active against Fasciola hepatica NEJs during the viability assessment after 24 hours: 1, 2, 3, 4, 5, 6, 7, 8, 9 and 10

The following compounds caused both a phenotype change and impaired motility significantly different to DMSO after 72 hours: 1, 2, 3, 4, 5, 6, 7, 8, and 9 and 10.

The following compounds were found to cause a Propidium Iodide positivity >than 90%: 2, 3, 4, 5, 6, 7, and 9.

Consequently, the following compounds displayed significant activity against juvenile stages of Fasciola hepatica: 1, 2, 3, 4, 5, 6, 7, 8 and 9.

TABLE 1

| | Score (mean) | Score (mean) |
|---|---|---|
| | phenotype 1 | motility 1 to 5 |
| Compound No. | to 6 (highest) | (highest) |
| Triclabendazole | 5.8 | 5.0 |
| DMSO | 1 | 1.3 |
| 1 | 5.5 | 5.0 |
| 2 | 5.8 | 5.0 |
| 3 | 5.7 | 5.0 |
| 4 | 5.7 | 5.0 |
| 5 | 5.7 | 5.0 |
| 6 | 5.6 | 5.0 |
| 7 | 5.7 | 4.8 |
| 8 | 5.7 | 5.0 |
| 9 | 5.7 | 5.0 |
| 10 | 3.8 | 4.8 |

Comparative GABA antagonist compounds that did not show antiflukicidal effect against Fasciola hepatica NEJs (showing a phenotypic score below 3) are shown in Table 2 below:

| Compound | |
|---|---|
| Number | Structure |
| C-11 | 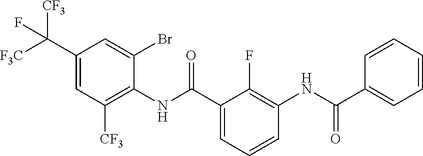 [SMILES: O=C(Nc1cccc(C(=O)Nc2c(Br)cc(C(F)(C(F)(F)F)C(F)(F)F)cc2C(F)(F)F)c1F)c1ccccc1] |
| C-12 | 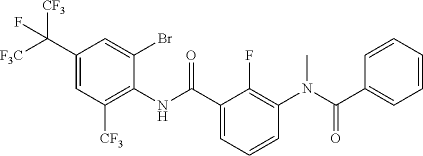 [SMILES: CN(C(=O)c1ccccc1)c1cccc(C(=O)Nc2c(Br)cc(C(F)(C(F)(F)F)C(F)(F)F)cc2C(F)(F)F)c1F] |
| C-13 | 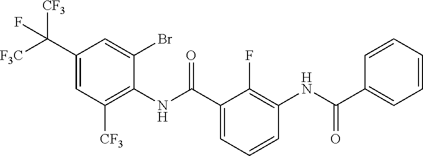 [SMILES: O=C(Nc1cccc(C(=O)Nc2c(Br)cc(C(F)(C(F)(F)F)C(F)(F)F)cc2C(F)(F)F)c1F)c1ccccc1] |
| C-14 | 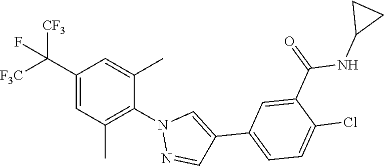 [SMILES: Cc1cc(C(F)(C(F)(F)F)C(F)(F)F)cc(C)c1-n1cc(-c2ccc(Cl)c(C(=O)NC3CC3)c2)cn1] |
| C-15 | 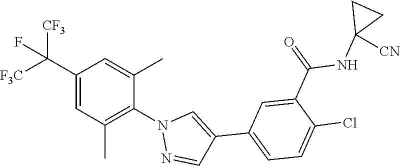 [SMILES: Cc1cc(C(F)(C(F)(F)F)C(F)(F)F)cc(C)c1-n1cc(-c2ccc(Cl)c(C(=O)NC3(C#N)CC3)c2)cn1] |
| C-16 | 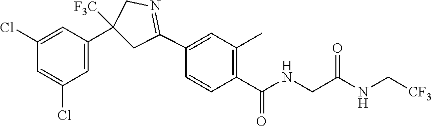 [SMILES: Cc1cc(C2=NCC(c3cc(Cl)cc(Cl)c3)(C(F)(F)F)C2)ccc1C(=O)NCC(=O)NCC(F)(F)F] |
| C-17 | 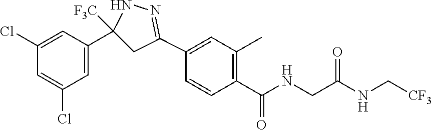 [SMILES: Cc1cc(C2=NNC(c3cc(Cl)cc(Cl)c3)(C(F)(F)F)C2)ccc1C(=O)NCC(=O)NCC(F)(F)F] |

### REFERENCES

[1] Edwards J, Brown M, Peak E, Bartholomew B, Nash R J, Hoffmann K F. The Diterpenoid 7-Keto-Sempervirol, Derived from Lycium chinense, Displays Anthelmintic Activity against both Schistosoma mansoni and Fasciola hepatica. PLoS neglected tropical diseases. 2015; 9(3):e0003604. Epub 2015/03/15. doi: 10.1371/journal.pntd.0003604. PubMed PMID: 25768432.

1. A method of treatment of fasciolosis in mammals comprising administering to a mammal in need there of an effective amount of a compound of Formula (I) 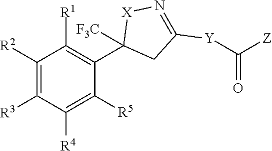
R¹-R⁵ is H, F, Cl or CF₃
X is O
Y is selected from 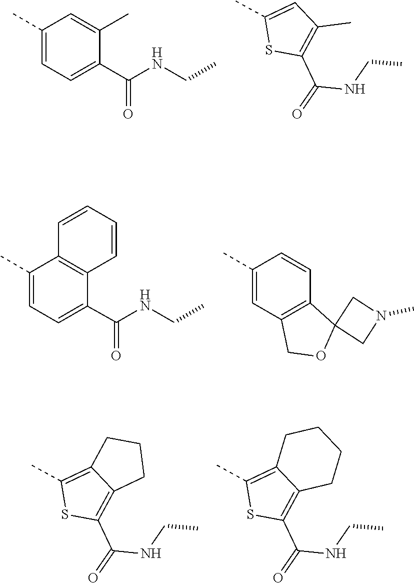
wherein [symbol]
denotes the bond to the isoxazoline [symbol]
denotes the bond to the carbonyl group
Z is selected from 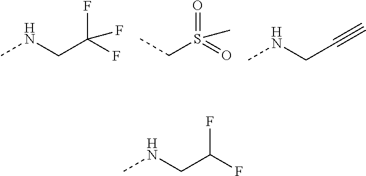
wherein [symbol]
denotes the bond to the carbonyl group.

2. The method according to claim 1 wherein the compound is selected from fluralaner, (S)-fluralaner, afoxolaner, sarolaner, or lotilaner.

3. (canceled)

4. The method of claim 1, wherein the mammal is selected from the group consisting of sheep and cattle.

5. The method of claim 1, wherein fasciolosis is treated during autumn, winter, or early spring season.

6. The method of claim 1, wherein the fasciolosis is due to an early immature stage liver fluke infection of the mammal by early immature larvae 1 to 6 weeks old.

7. The method of claim 1, wherein the fasciolosis is due to an immature stage liver fluke infection of the mammal by immature larvae 6 to 9 weeks old.

8. The method of claim 1, wherein the fasciolosis is due to a mature stage liver fluke infection of the mammal by mature flukes >9 weeks old.

9. The method of claim 1 wherein the compound of Formula (I) is administered in a composition comprising a pharmaceutically acceptable carrier.

10. The method of claim 9 wherein the composition further comprises an additional active ingredient.

11. The method of claim 9, wherein the composition is administered orally to the mammal.

12. The method of claim 9, wherein the composition is administered subcutaneously to the mammal.

13. The method according to claim 9 wherein the mammal is selected from the group consisting of sheep and cattle.

14. The method according to claim 9, wherein fasciolosis is treated during autumn, winter, or early spring season.

15. The method according to claim 9, wherein the fasciolosis is due to an early immature stage liver fluke infection of the mammal by early immature larvae 1 to 6 weeks old.

16. The method according to claim 9, wherein the fasciolosis is due to an immature stage liver fluke infection of the mammal by immature larvae 6 to 9 weeks old.

17. The method according to claim 9, wherein the fasciolosis is due to a mature stage liver fluke infection of the mammal by mature flukes >9 weeks old.

18. A method of treatment of fasciolosis in mammals comprising administering to a mammal in need there of an effective amount of the compound tigolaner.

19. The method of claim 18 wherein the tigolaner is administered in a composition comprising a pharmaceutically acceptable carrier.

20. The method of claim 19 wherein the composition further comprises an additional active ingredient.
